(19) United States
(12) Patent Application Publication
(10) Pub. No.: US 2024/0058299 A1
(43) Pub. Date: Feb. 22, 2024

(54) GENISTEIN TREATMENT OF INFLAMMATORY AND IMMUNOLOGICAL DISORDERS

(71) Applicant: Humanetics Corporation, Minneapolis, MN (US)

(72) Inventors: Artur A. Serebrenik, Philadelphia, PA (US); Michael D. Kaytor, Maplewood, MN (US); John C. Dykstra, Edina, MN (US); Ronald J. Zenk, Shorewood, MN (US); Brett D. Anderson, Minneapolis, MN (US)

(21) Appl. No.: 18/271,125

(22) PCT Filed: Jan. 11, 2022
(86) PCT No.: PCT/US2022/011964

§ 371 Date: Jul. 6, 2023

Related U.S. Application Data
(60) Provisional application No. 63/255,249, filed on Oct. 13, 2021.
(60) Provisional application No. 63/136,024, filed on Jan. 11, 2021.

(51) Int. Cl. A61K 31/352; A61P 29/00; A61K 47/32

(52) U.S. Cl. CPC A61K 31/352; A61P 29/00; A61K 47/32

(57) ABSTRACT

Methods and materials and methods for reducing organ and tissue damage associated with inflammatory and immunological disorders are provided herein. For example, this document provides methods and materials for using genistein to treat respiratory distress syndrome or acute lung injury (e.g., pneumonitis, pulmonary fibrosis, dyspnea, pneumonia, and/or pulmonary edema resulting from viral infection, allergic responses, chemical insults, radiation, tissue injury, persistent infections, or autoimmune reactions).

### CROSS-REFERENCE TO RELATED APPLICATIONS

This application claims priority from U.S. Provisional Application Ser. No. 63/136,024, filed Jan. 11, 2021, and U.S. Provisional Application Ser. No. 63/255,249, filed Oct. 13, 2021. The disclosures of the prior applications are considered part of (and are incorporated by reference in) the disclosure of this application.

### STATEMENT AS TO FEDERALLY SPONSORED RESEARCH

This invention was made with government support under HHSN272201800011C, awarded by the National Institutes of Health. The government has certain rights in the invention.

### TECHNICAL FIELD

This document relates to materials and methods for reducing organ and tissue damage associated with inflammatory and immunological disorders.

### BACKGROUND

Fibrosis, which involves progressive scarring or thickening of tissue, can be an end result of chronic inflammatory reactions induced by a variety of stimuli, including allergic responses, chemical insults, radiation, tissue injury, persistent infections, and autoimmune reactions. Examples of diseases with a pathological feature of fibrosis include pancreatitis, myocarditis, acute respiratory distress syndrome, and severe acute respiratory syndrome (SARS), a viral respiratory illness caused by the SARS-associated coronavirus (SARS-CoV). SARS-CoV caused a global epidemic in 2002 and 2003, with more than 8000 confirmed cases in more than 25 countries. SARS-CoV-2 is a novel coronavirus that was first reported in December 2019 in Wuhan, China, and resulted in a global pandemic with substantial morbidity and mortality (Zou et al., N Engl J Med 382:1177-1179, 2020).

### SUMMARY

This document is based, at least in part, on the discovery that genistein can be used to treat mammals identified as having inflammatory and/or immunological disorders. For example, genistein's ability to ameliorate pneumonitis and pulmonary fibrosis makes it useful as a therapeutic in COVID-19 patients, and particularly in patients suffering from the more severe, lung-related effects of the disease. Genistein's ability to reduce inflammation and associated tissue damage also makes it useful as a therapeutic in other disorders, such as pancreatitis, myocarditis, non-alcoholic fatty liver disease, non-alcoholic steatohepatitis, and asthma, among others.

In one aspect, this document features a method for reducing inflammation and/or an immune response in a mammal identified as having an inflammatory or immunological disorder. The method can include administering to the mammal a composition containing genistein in an amount effective to reduce inflammation and/or an immune response in the mammal. The genistein can be nanoparticulate genistein. The composition can have a nanoparticulate genistein concentration between about 250 mg/mL and about 500 mg/mL. The mammal can be a human. The mammal can be identified as having an inflammatory disorder selected from the group consisting of pancreatitis, myocarditis, and acute respiratory distress syndrome. In some cases, the inflammatory or immunological disorder is not COVID-19. The composition can contain nanoparticulate genistein with a particle size distribution characterized by a d(0.5) less than or equal to 0.5 μm. The composition can further contain one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The composition can contain a nonionic surfactant, where the nonionic surfactant is present in an amount ranging from about 0.01% to about 10% by weight (w/w). The amount of water soluble polymer can be about 0.5% to about 15% (w/w). The composition can contain a diluent and a preservative. The composition can further contain a non-ionic surfactant. The composition can contain nanoparticulate genistein in an amount ranging up to about 50% (w/w), or in an amount of about 20% to about 35% (w/w). The composition can contain nanoparticulate genistein at a concentration of about 325 mg/mL. The composition can have a pH of about 2 to about 12. The composition can be formulated as a tablet, a capsule, a gel cap, a powder, or a liquid suspension. The method can include administering the composition orally, intramuscularly, subcutaneously, or intravenously. The method can include administering the composition within about 24 hours to about 7 days of diagnosis of the mammal as having the inflammatory or immunological disorder, or within about 24 hours to about 7 days of onset of one or more symptoms of the inflammatory or immunological disorder. The method can include administering the composition at least once daily. The method can include administering the composition for about 1 week to about 12 weeks. The method can include administering the composition in an amount of about 0.5 g to about 2.5 g per dose. The method can include administering the composition in an amount of about 1 g to about 1.5 g per dose.

This document also features a method for reducing pneumonitis, pulmonary fibrosis, dyspnea, pneumonia, and/or pulmonary edema in a mammal identified as being or as having been infected with a coronavirus. The method can include administering to the mammal a composition containing genistein in an amount effective to reduce pneumonitis, pulmonary fibrosis, dyspnea, pneumonia, and/or pulmonary edema in the mammal. The genistein can be nanoparticulate genistein. The composition can have a nanoparticulate genistein concentration between about 250 mg/mL and about 500 mg/mL. The mammal can be a human. The mammal can be identified as being infected with SARS-Cov-2. The composition can contain nanoparticulate genistein with a particle size distribution characterized by a d(0.5) less than or equal to 0.5 μm. The composition can further contain one or more pharmaceutically acceptable excipients forming a suspension medium, where the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The amount of water soluble polymer can be about 0.5% to about 15% (w/w). The composition can contain a diluent and a preservative. The composition further can contain a non-ionic surfactant. The composition can contain nanoparticulate genistein in an amount ranging up to about 50% (w/w) [e.g., an amount of about 20% to about 35% (w/w)]. The composition can contain nanoparticulate genistein at a concentration of about 325 mg/mL. The composition can have a pH of about 2 to about 12. The composition can be formulated as a tablet, a capsule, a gel cap, a powder, or a liquid suspension. The method can include administering the composition orally, intramuscularly, subcutaneously, or intravenously. The method can include administering the composition within about 1 to about 96 hours of diagnosis of the mammal as having a coronavirus infection or within about 1 to 96 hours of onset of one or more symptoms of coronavirus infection. The method can include administering the composition beginning within about 1 hour to about 72 hours of diagnosis of the mammal as having pneumonitis, pneumonia, or pulmonary fibrosis. The method can include administering the composition at least once daily. The method can include administering the composition in an amount of about 0.5 g to about 2.5 g per dose. The method can include administering the composition in an amount of about 1 g to about 1.5 g per dose.

In another aspect, this document features the use of a composition comprising genistein to reduce inflammation and/or an immune response in a mammal identified as having an inflammatory or immunological disorder. The genistein can be nanoparticulate genistein. The composition can have a nanoparticulate genistein concentration between about 250 mg/mL and about 500 mg/mL. The mammal can be a human. The mammal can be identified as having an inflammatory disorder selected from the group consisting of pancreatitis, myocarditis, and acute respiratory distress syndrome. In some cases, the inflammatory or immunological disorder is not COVID-19. The composition can contain nanoparticulate genistein with a particle size distribution characterized by a d(0.5) less than or equal to 0.5 μm. The composition can further contain one or more pharmaceutically acceptable excipients forming a suspension medium, where the one or more pharmaceutically acceptable excipients include a water soluble polymer comprising a polyvinylpyrrolidone. The one or more pharmaceutically acceptable excipients can include a nonionic surfactant, a diluent, or a buffer. The composition can contain a nonionic surfactant, where the nonionic surfactant is present in an amount ranging from about 0.01% to about 10% by weight (w/w). The amount of water soluble polymer can be about 0.5% to about 15% (w/w). The composition can contain a diluent and a preservative. The composition can further contain a non-ionic surfactant. The composition can contain nanoparticulate genistein in an amount ranging up to about 50% (w/w). The composition can contain nanoparticulate genistein in an amount of about 20% to about 35% (w/w). The composition can contain nanoparticulate genistein at a concentration of about 325 mg/mL. The composition can have a pH of about 2 to about 12. The composition can be formulated as a tablet, a capsule, a gel cap, a powder, or a liquid suspension. The composition can be formulated for oral, intramuscular, subcutaneous, or intravenous administration. The composition can be for use within about 24 hours to about 7 days of diagnosis of the mammal as having the inflammatory or immunological disorder, or within about 24 hours to about 7 days of onset of one or more symptoms of the inflammatory or immunological disorder. The composition can be for use at least once daily, or for use for about 1 week to about 12 weeks. The composition can be formulated for administration in an amount of about 0.5 g to about 2.5 g per dose, or for administration in an amount of about 1 g to about 1.5 g per dose.

In another aspect, this document features the use of a composition comprising genistein to reduce pneumonitis, pulmonary fibrosis, dyspnea, pneumonia, and/or pulmonary edema in a mammal identified as being or having been infected with a coronavirus. The genistein can be nanoparticulate genistein. The composition can have a nanoparticulate genistein concentration between about 250 mg/mL and about 500 mg/mL. The mammal can be a human. The mammal can be identified as being infected with SARS-Cov-2. The composition can contain nanoparticulate genistein with a particle size distribution characterized by a d(0.5) less than or equal to 0.5 μm. The composition can further contain one or more pharmaceutically acceptable excipients forming a suspension medium, wherein the one or more pharmaceutically acceptable excipients include a water soluble polymer comprising a polyvinylpyrrolidone. The one or more pharmaceutically acceptable excipients can include a nonionic surfactant, a diluent, or a buffer. The nonionic surfactant can be present in an amount ranging from about 0.01% to about 10% by weight (w/w). The amount of water soluble polymer can be about 0.5% to about 15% (w/w). The composition can contain a diluent and a preservative. The composition can further contain a non-ionic surfactant. The composition can contain nanoparticulate genistein in an amount ranging up to about 50% (w/w). The composition can contain nanoparticulate genistein in an amount of about 20% to about 35% (w/w). The composition can contain nanoparticulate genistein at a concentration of about 325 mg/mL. The composition can have a pH of about 2 to about 12. The composition can be formulated as a tablet, a capsule, a gel cap, a powder, or a liquid suspension. The composition can be for oral, intramuscular, subcutaneous, or intravenous administration. The composition can be for administration within about 1 to about 96 hours of diagnosis of the mammal as having a coronavirus infection or within about 1 to 96 hours of onset of one or more symptoms of coronavirus infection. The composition can be for administration beginning within about 1 hour to about 72 hours of diagnosis of the mammal as having pneumonitis, pneumonia, or pulmonary fibrosis. The composition can be for administration at least once daily. The composition can be formulated for administration in an amount of about 0.5 g to about 2.5 g per dose, or for administration in an amount of about 1 g to about 1.5 g per dose.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show that a composition containing an oral suspension of nanoparticulate genistein was able to mitigate the onset of radiation-induced edema and fibrosis in the lung. Mice were exposed to 11.0 Gy whole thoracic lung irradiation (WTLI) or sham irradiation and then treated with vehicle or genistein suspension (400 mg/kg) once daily for 2, 4, or 6 weeks. FIG. 1A includes representative images of animal lungs stained with Masson's Trichrome. Images on the left are whole-lung, and images on the right are high magnification (10×). Unirradiated lungs showed well-aerated alveoli. Irradiated lungs of untreated mice displayed significant morphological changes, with almost complete loss of airways due to edema, congestion, and fibrotic scarring. The irradiated lungs of mice treated with genistein suspension (400 mg/kg) for 4 weeks showed significantly less tissue damage. For example, an absence of perivascular lymphocytic infiltration (arrow) was observed in treated mice. FIG. 1B is a graph plotting mean wet lung weight, showing that irradiated mice treated daily with genistein suspension for 2 weeks starting 24 hours after irradiation had reduced mean wet lung weight as compared to animals that received radiation alone (p<0.05). Error bars±SEM. FIG. 1C is a graph plotting the reduction in levels of fibrosis (determined by Masson's Trichrome staining, with scoring as described by Ashcroft et al., J Clin Pathol 41:467-470, 1988) when genistein suspension was administered starting 24, 72, or 120 hours after radiation exposure and continued daily for 2 weeks.

FIGS. 2A-2C show that an oral suspension of nanoparticulate genistein mediated radioprotection of normal lung tissue in a mouse xenograft model. FIG. 2A is an image of a mouse in which A549 cells were implanted subcutaneously, followed by treatment with 12.5 Gy of radiotherapy (RT). Genistein suspension (200 mg/kg/d) was given by oral gavage beginning 7 days prior to whole thorax-lung irradiation (WTLI, RT), and continued daily until the end of the study. The white box indicates the path of radiation, and the circle indicates the tumor site. FIG. 2B contains representative lung tissue H&E images from the control (No RT), irradiated (RT), and genistein-treated (Genistein+RT) groups. FIG. 2C is a graph plotting wet weight of lungs from WTLI-treated animals after administration of vehicle or genistein, as an indicator of pneumonitis.

FIGS. 3A and 3B are graphs plotting pulmonary adverse event incidence rates in non-small cell lung cancer (NSCLC) clinical trials. The average incidence rate of dyspnea (FIG. 3A) and pulmonary fibrosis (FIG. 3B) in historical patients (Bradley et al., Lancet Oncol 16(2):187-199, 2015; solid bars; N=151) and genistein phase 1b/2a trial (white bars; N=21) are plotted.

FIG. 4A is a graph plotting survival of microHeart cells (human induced pluripotent stem cell-derived cardiac cells) that were treated with vehicle or genistein at the indicated concentrations, and then subjected to γ-irradiation 24 hours later. Cell survival was measured by trypan blue staining. The number of surviving cells 24 hours after irradiation is plotted. Genistein treatment was radioprotective. FIG. 4B is a graph plotting survival of microHeart cells that were treated with vehicle or 0.5 μM genistein, and then exposed to mixed field radiation (˜32% neutron, −68% gamma) 24 hours later. Cell survival was measured by trypan blue staining. The number of surviving cells 72 hours after irradiation is plotted. Again, genistein was radioprotective.

FIG. 5 is an image showing a western blot of total protein lysates from heart and lung tissue harvested from mice that were treated with vehicle or genistein and subjected to high linear energy transfer (LET) proton irradiation. Mice were treated with a single subcutaneous injection of nanoparticulate genistein (200 mg/kg) 24 hours prior to radiation exposure. Tissue samples were collected 72 hours post radiation exposure. Lysates were stained with antibodies against p53, γH2AX, and tubulin (control).

### DETAILED DESCRIPTION

This document provides methods and materials for treating mammals identified as having inflammatory disorders and/or immunological disorders. Left unchecked, inflammatory responses and immune responses can have a number of detrimental effects. For example, fibrosis—progressive scarring or thickening of tissue—is an end result of chronic inflammatory reactions induced by a variety of stimuli, including allergic responses, chemical insults, radiation, tissue injury, persistent infections, and autoimmune reactions. In some cases, the immune system can cause an uncontrolled and excessive release of pro-inflammatory signaling molecules, or cytokines. The resulting “cytokine storm” can cause multisystem organ failure and death. A number of infectious and non-infectious agents can cause cytokine storms. Infectious agents that can cause cytokine storm include, for example, viruses (e.g., influenza viruses such as H5N1 influenza, coronaviruses such as SARS-CoV-1 and SARS-CoV-2, Epstein-Barr virus, and cytomegalovirus) and bacteria (e.g., group A streptococcus).

Disorders that can be treated using the materials and methods described herein include, without limitation, respiratory diseases caused by coronaviruses. For example, SARS-CoV-2, which causes the disease known as COVID-19, is thought to rapidly invade and destroy human lung cells. The destroyed cells fill patients' airways with debris and fluid, leading to shortness of breath and triggering an immune response that results in an influx of cytokines, which can further exacerbate lung inflammation and respiratory distress. Thus, patients with severe cases of COVID-19 often present with severe pulmonary dysfunction, including shortness of breath, pulmonary edema, pneumonia, pneumonitis, and/or pulmonary fibrosis.

Coronaviruses are RNA viruses that typically cause respiratory tract infections, sometimes lethally. For example, SARS-CoV and MERS-CoV are responsible for severe acute respiratory syndrome (SARS) and middle east respiratory syndrome (MERS), respectively, both of which can be deadly. The SARS-CoV-2 virus was first identified in patients exhibiting pneumonia and shortness of breath. SARS-CoV-2 enters host cells by binding the cellular receptor, angiotensin-converting enzyme 2 (ACE2), which is highly expressed in the epithelial cells of the airways and lungs (Jia et al., J Virol 79(23):14614-14621, 2005). Once SARS-CoV-2 infects cells, it triggers an immune response that involves recruitment of macrophages and neutrophils into the lungs, and the release of a pro-inflammatory cytokine storm. Cytokines that commonly are overexpressed in the serum of patients with SARS or COVID-19 include interleukin-6 (IL-6), interleukin-8 (IL-8), tumor necrosis factor alpha (TNFα), interferon gamma (INF-γ), and transforming growth factor beta (TGFβ) (Dosch et al., Virus Res 142(1-2):19-27, 2009; Guo et al., Mil Med Res 7(1):11, 2020; and Lin et al., Emerg Microbes Infect 9(1):727-732, 2020). In serious cases of COVID-19, such as those that require hospitalization and mechanical ventilation, the severe immune response triggered in patients can result in acute respiratory distress syndrome (ARDS). In one study of 138 hospitalized patients with COVID-19, the median time until ARDS developed was eight days (Wang et al., JAMA 323(11):1061-1069, 2020). Infiltration of immune cells (particularly neutrophils) into the lungs is important because they produce factors that can inhibit viral replication and destroy infected cells to prevent further production of viral progeny (Galani and Andreakos, J Leukoc Biol 98(4):557-564, 2015). During ARDS, however, neutrophils and other immune cells contribute to lung injury by releasing toxic oxygen radicals and proteases that damage epithelial and endothelial cells, which results in increased alveolar permeability (Crimi and Slutsky, Best Pract Res Clin Anaesthesiol 18(3):477-492, 2004). In the acute phase of disease, this damage causes pulmonary edema and a potentially fatal decrease in respiratory function. In the later stage of disease, inflammation results in the over production of pro-fibrotic factors and collagen production, which leads to stiffening of the lungs and fibrosis. The pathophysiology of ARDS is not specific to SARS-CoV-2 infection, and is similar across other respiratory infections or pulmonary traumas (Reiss et al., Curr Opin Crit Care 24(1):1-9, 2018).

Pulmonary fibrosis due to ARDS is a primary reason that patients who recover from SARS have a lower quality of life and lower respiratory function (Wang et al., Zhonghua Shao Shang Za Zhi 36(0):E006, 2020, doi 10.3760/cma.j.cn501120-20200307-00132). Studies of patients with SARS showed that 45% had pulmonary fibrosis one month after infection, while 28% had pulmonary fibrosis twelve months after infection (Venkataraman and Frieman, Antiviral Res 143:142-150, 2017). Case studies of patients hospitalized with severe COVID-19 indicated that some developed pulmonary fibrosis as early as the first week of infection, and the percentage of patients with pulmonary fibrosis ballooned by the second or third week of infection (Zhang, Intensive Care Med 2020, doi 10.1007/s00134-020-05990-y; and Xiong et al., Invest Radiol 2020, doi 10.1097/RLI.0000000000000674). In addition, studies in a transgenic mouse that expresses human ACE2 in the lungs showed that the animals developed pneumonitis within three days of infection with SARS-CoV-2, and developed pulmonary fibrosis within five days of infection (Bao et al., bioRxiv 2020:2020.02.07.939389, doi 10.1101/2020.02.07.939389).

In some cases, the inflammatory or immunological disorder that is treated by a method provided herein is not COVID-19. The methods provided herein can be used to treat any other appropriate disorder characterized by inflammation and/or improper immune responses. Examples of such disorders are listed below.

Acute and chronic pancreatitis are characterized by inflammation of the pancreas due to autodigestion of pancreatic tissue by aberrant activation of digestive enzymes produced in the pancreas. These disorders are commonly caused by gallstone blockage of the common bile duct downstream of the pancreatic duct junction, and by heavy alcohol use. Other causes include infection (e.g., viral, bacterial, fungal, or parasitic infection), medications (e.g., statins or ACE inhibitors), and trauma. In some cases, pancreatitis (e.g., acute pancreatitis) can be accompanied by systemic inflammatory response syndrome (SIRS).

Myocarditis relates to inflammation of the myocardium. Myocarditis is most commonly caused by viral infection (e.g., by coxsackievirus or adenovirus), but also can be caused by bacterial, fungal, and parasitic infections. Other etiologies include prescription drugs (e.g., anthracyclines or chemotherapeutics) and autoimmune disease (e.g., scleroderma or lupus).

Crohn's disease is characterized by chronic inflammation of the gastrointestinal tract. This disease is associated with dysregulation of the gut microbiome composition. The underlying cause is not known, but there is clearly a strong, heterogeneous genetic component. Environmental factors that can contribute to the risk of developing Crohn's disease include smoking and diet.

Ulcerative colitis results from chronic inflammation of the colon and rectum, leading to the formation of ulcers. Ulcerative colitis is thought to be potentiated by an aberrant immune response to commensal microbes. Genetic and environmental factors may have a significant impact on the risk of developing this disease.

Metabolic syndrome is defined by the coincidence of three of the five underlying conditions: abdominal obesity, hypertension, hyperglycemia, high serum triglyceride levels, and low serum HDL levels. Metabolic syndrome can be predictive of the risk of developing Type 2 diabetes and cardiovascular disease. The underlying etiology likely is a combination of diet, lifestyle, genetics, and environmental factors.

Ischemic reperfusion injury is caused by reperfusion of a tissue following a period of ischemia due to cessation of blood flow (e.g., due to stroke, myocardial infarction, or a surgical procedure), which may result in the release of proinflammatory cytokines and other proinflammatory mediators due to cell death, which may promote further damage to affected tissue.

Non-alcoholic fatty liver disease (NAFLD) is characterized by excessive fat accumulation in the liver that is not attributed to a secondary cause, such as alcohol consumption or viral hepatitis. Genetics and diet appear to be the primary risk factors for NAFLD. In some cases, NAFLD can progress to non-alcoholic Steatohepatitis (NASH), which is characterized by chronic inflammation of the liver and varying degrees of liver fibrosis.

Fibromyalgia is characterized by chronic, widespread pain, fatigue, cognitive dysfunction and sleep issues. The cause and underlying pathology of the disease is not well understood, although genetics, inflammatory mechanisms, and physical and emotional stress appear to play a role.

Rheumatoid arthritis is an autoimmune disease characterized by chronic inflammation within the joints. The cause of this disorder is unknown, but genetics, being female, and smoking appear to contribute to the overall risk of developing rheumatoid arthritis.

Psoriasis is characterized by raised, itchy, and scaly patches of skin due to unusually rapid proliferation of keratinocytes. Psoriasis is an autoimmune disease that appears to be driven aberrant stimulation of immature keratinocytes by dendritic cells, T cells and macrophages. The cause is not known, but genetics appear to play a significant role in overall risk. Stress and infections (particularly strep throat) are commonly associated with flare-ups of the disease.

Asthma is characterized by long-term inflammation of the bronchi and bronchioles, which can trigger the narrowing of airways due to smooth muscle contraction. The underlying cause is not well understood, but genetics and environmental factors appear to be the main contributors. The hygiene hypothesis postulates that reduced exposure to non-pathogenic bacteria and viruses at an early age may lead to a heightened sensitivity to benign antigens later in development, which may play a role in the development and persistence of asthma.

Scleroderma is a chronic autoimmune disease characterized by progressive fibrosis that may affect the skin, connective tissues, and internal organs. The overall cause is unknown, and there are limited genetic associations with the disease.

Cytomegalovirus infection, exposure to organic solvents, and a maladaptive immune response to circulating fetal cells in the mother during pregnancy have been postulated as potential initiators of this autoimmune phenomenon.

Encephalitis is, broadly, inflammation of the brain. This disease is most frequently caused by viral infection (e.g., herpes simplex virus infection), but bacterial, fungal and parasitic infections, as well as autoimmunity, also are recognized initiators.

Atherosclerosis is characterized by the gradual buildup of atheromatous plaques on the walls of arteries. Plaque deposits are nucleated by lesions formed on the endothelium, which appear the product of adherent monocytes proliferating and differentiating into macrophages. The disease is generally asymptomatic until narrowing the artery results in the loss of blood flow due to a trapped blood clot. High levels of LDL, hypertension, diabetes, smoking and obesity are recognized risk factors for atherosclerosis.

Multiple sclerosis is an autoimmune disease characterized by demyelination of nerve cells, leading to progressive loss of nervous system function. The cause of the disease is unknown, but genetics and environmental factors likely contribute to overall risk. Several pathogens (e.g., Epstein-Barr virus and herpes simplex virus) have been proposed as initiators of this autoimmunity.

Idiopathic pulmonary fibrosis (IPF) is an interstitial lung disease characterized by chronic inflammation, accompanied by an uncontrolled healing response that causes fibrosis of tissues between the lung's alveoli.

Acute kidney injury can be associated with intrarenal and/or systemic inflammation. A sudden drop in blood flow to the kidneys, various medicines, poisons, infections, or a blockage that stops urine flow can cause acute kidney injury. Underlying inflammation can increase the risk of developing acute kidney injury.

Chronic kidney disease can result from chronic inflammation, which can lead to scarring (fibrosis) in the glomeruli. Patients with chronic kidney disease often display a chronic increase in markers of inflammation, which can be intensified by disease progression and the onset of hemodialysis.

As described herein, compositions containing genistein can be used as medical countermeasures or therapeutics for reducing inflammation, immune responses, and resulting sequelae in mammals with inflammatory and/or immune disorders (e.g., pneumonitis and/or pulmonary fibrosis in COVID-19 patients). Genistein [5,7-dihydroxy-3-(4-hydroxyphenyl)-4H-chromen-4-one (USAN); 5,7-dihydroxy-3-(4-hydroxyphenyl)-chromen-4-one (IUPAC); 5,7-dihydroxy-3-(4-hydroxyphenyl)-4H-1-benzopyran-4-one; 5,7,4′-trihydroxyisoflavone; or 4′,5,7-trihydroxyisoflavone) is a phytoestrogen in the category of isoflavones. Genistein's chemical structure is shown in Formula (1):

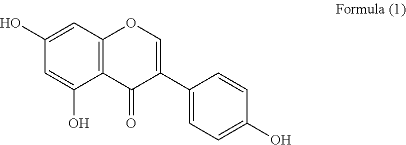 [SMILES: O=c1c(-c2ccc(O)cc2)coc2cc(O)cc(O)c12]

Genistein is one of several known isoflavones that are normally found in plants. The main sources of natural genistein are soybeans and other legumes. Genistein also is commercially available, and may be obtained in synthetic, purified form (e.g., from DSM Nutritional Products, Inc., Parsippany, NJ).

Genistein has the ability to reduce expression of NF-κB, pro-inflammatory cytokines [e.g., IL-1, IL-6, and TNFα), and pro-fibrotic proteins (e.g., TGFβ, matrix metalloproteinase-2 (MMP-2), matrix metalloproteinase-9 (MMP-9) and lactate dehydrogenase (LDH)] (see, Day et al., J Rad Res 49(4):361-372, 2008; Ha et al., Rad Res 180(3):316-325, 2013; Ji et al., PloS One 7(12):e53101, 2012; Kang et al., Crit Care Med 31(2):517-524, 2003; Kang et al., Am J Resp Crit Care Med 164(12):2206-2212, 2001; Kim et al., Int Journal Mol Med 34(6):1669-1674, 2014; Morris et al., Rad Res 135(3):320-331, 1993; and Zhu et al., Biosci Biotechnol Biochem 84(3):544-551, 2020), all likely occurring via activation of estrogen receptor beta (ERβ) and downstream repression of NF-κB. Genistein's effects on NF-κB, pro-inflammatory cytokines, and pro-fibrotic proteins are evidence of its anti-fibrotic and anti-inflammatory properties. Data providing further evidence of genistein's effects are discussed in the Examples below. Thus, compositions containing genistein can be used to reduce or prevent pulmonary fibrosis, pneumonitis, and/or other adverse lung events (e.g., dyspnea, pulmonary edema, and/or pneumonia) in subjects identified as having, being at risk of having, or at risk of exposure to any coronavirus infection, including COVID-19.

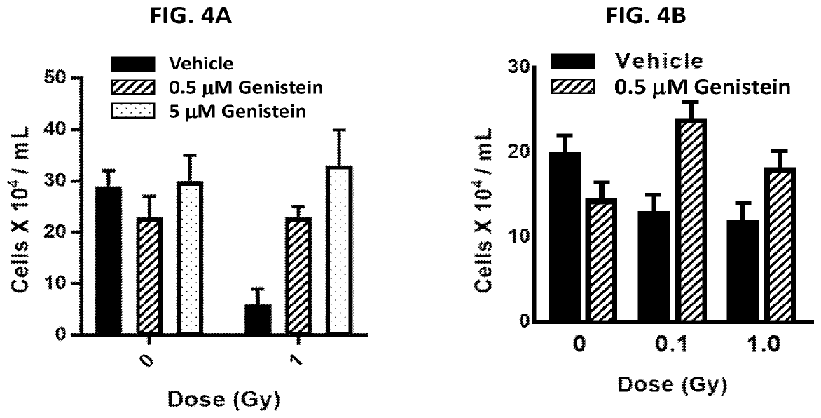

Any appropriate genistein-containing composition can be used to alleviate inflammation, immune response, and/or resulting sequelae (e.g., fibrosis and/or other tissue damage, such as endothelial apoptosis, necrosis, and diffuse alveolar damage in the lung) in various tissues and organs, allowing for improved function of the affected organ(s). In the lung, for example, a genistein-containing composition can be used to treat pneumonitis, pulmonary fibrosis, dyspnea, pneumonia, and/or pulmonary edema, as described herein. In some cases, a composition can include genistein nanoparticles, which can have improved oral and/or parenteral bioavailability as compared to genistein that is not in nanoparticle form. Nanoparticle formulations can contain sub-micron size genistein particles, which can be manufactured using a wet nanomilling process that reduces genistein to a median particle size of less than 0.2 μm. See, e.g., U.S. Pat. No. 8,551,530. Pharmacokinetic experiments using such a genistein nanosuspension in mice demonstrated dramatically increased oral bioavailability as compared to formulations containing non-micronized genistein. See, FIGS. 4-7 of U.S. Pat. No. 8,551,530.

In some embodiments, a composition can contain genistein (e.g., nanoparticulate genistein or genistein that is not in nanoparticle form), at a concentration between about 100 mg/mL and about 500 mg/mL (e.g., about 100 mg/mL to about 400 mg/mL, about 150 mg/mL to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL). Compositions containing nanoparticulate genistein can have a particle size distribution characterized by a median diameter [d(0.5)] that is less than or equal to 0.5 μm (e.g., less than or equal to 0.4 μm, less than or equal to 0.3 μm, or less than or equal to 0.2 μm). The composition also can contain one or more other components, as described herein (e.g., one or more pharmaceutically acceptable excipients that form a suspension medium, such as a water soluble polymer, a nonionic surfactant, a diluent, or a buffer). In some embodiments, the suspension medium may be non-aqueous, such as edible lipids, oils, and fats from plant and animal sources (e.g., olive, corn, soy, marine, coconut, palm, palm kernel, cotton seed, peanut, safflower, sesame, sunflower, almond, cashew, macadamia, pecan, pine nut, walnut, lemon, orange, flax seed, and borage oils).

In some cases, the genistein compositions used in the methods provided herein can be formulations that include genistein in a solution along with one or more pharmaceutically acceptable carriers, excipients, and/or diluents. In some cases, the genistein-containing compositions used in the methods provided herein can be suspension formulations that include genistein (e.g., nanoparticulate genistein) suspended in a medium containing one or more pharmaceutically acceptable carriers, excipients, and/or diluents. Pharmaceutically acceptable carriers, excipients, and diluents suitable for therapeutic use include those described, for example, in Remington's Pharmaceutical Sciences, Maack Publishing Co. (A. R. Gennaro (ed.), 1985). In some cases, polyethylene glycol (PEG) can be used as a carrier in a composition that also contains genistein that is not in nanoparticle form.

In some cases, genistein compositions can include a suspension containing nanoparticulate genistein suspended in a non-aqueous medium, such as an edible plant or animal oil (e.g., olive oil, sunflower oil, corn oil, soy oil, marine oil, coconut oil, palm oil, palm kernel oil, cotton seed oil, safflower oil, sesame oil, peanut oil, almond oil, cashew oil, pecan oil, pine nut oil, macadamia oil, orange oil, flax seed oil, lemon oil, walnut oil, borage oils, fish oils, and dairy derived fats). See, e.g., U.S. Pat. No. 9,084,726.

Genistein compositions can, in some cases, include a suspension containing nanoparticulate genistein suspended in a medium including one or more water soluble polymers and one or more nonionic surfactants. See, e.g., U.S. Pat. No. 8,551,530. Nonionic surfactants can facilitate wetting and aid in preventing agglomeration of genistein particles (e.g., nanoparticulate genistein). Suitable nonionic surfactants include, without limitation, polysorbates, poloxamers, polyoxyethylene castor oil derivatives, bile salts, lecithin, 12-hydroxystearic acid-polyethylene glycol copolymer, and the like. In some embodiments, a genistein composition can include a nonionic surfactant selected from the group consisting of polysorbate 80 (TWEEN® 80), polysorbate 20 (TWEEN® 20), Poloxamer 188, and combinations thereof. In some cases, the total nonionic surfactant content in a genistein composition can range from about 0.01% to about 10% by weight (w/w) (e.g., about 0.2% to about 5% (w/w), about 0.2% to about 2% (w/w), about 0.2% to about 1% (w/w), about 0.2% to about 0.6% (w/w), and about 0.2% to about 0.8% (w/w).

Water soluble polymers can serve to enhance the viscosity of a suspension and/or to stabilize genistein particles (e.g., nanoparticulate genistein) against particle agglomeration or potential deleterious effects from other formulation components, for example. Water soluble polymers are pharmaceutically acceptable polymers that can be dissolved or dispersed in water. Suitable water soluble polymers include, without limitation, vegetable gums (e.g., alginates, pectin, guar gum, and xanthan gum), modified starches, polyvinylpyrrolidone (PVP), hypromellose (HPMC), methylcellulose, and other cellulose derivatives (e.g., sodium carboxymethylcellulose, hydroxypropylcellulose, and the like). In some cases, the genistein compositions described herein can include a poloxamer (e.g., Poloxamer 188) as a water soluble polymer. Poloxamer 188 is both a polymer and surfactant. The total water soluble polymer content in a genistein composition as provided herein can range from about 0.5% to about 15% (w/w) [e.g., about 1% to about 10% (w/w), about 10% to about 15% (w/w), about 12% to about 15% (w/w), about 1% to about 8% (w/w), and about 1% to about 5% (w/w)].

Carriers suitable for use in the genistein formulations described herein also include pharmaceutically acceptable aqueous carriers such as, sterile water, physiologically buffered saline, Hank's solution, and Ringer's solution. The formulations also can contain one or more buffers [e.g., one or more citrate buffers, phosphate buffers, tris(hydroxymethyl)aminomethane (TRIS) buffers, and/or borate buffers], to achieve a desired pH and osmolality. Pharmaceutical formulations typically have a pH in the range of about 2 to about 12. In some embodiments, the genistein formulations provided herein can have a pH that falls in a range that more closely approximates physiologic pH (e.g., about 4 to about 8, or about 5 to about 7).

The genistein compositions used in the methods described herein also can, in some cases, include one or more diluents. Suitable diluents include those selected from, without limitation, pharmaceutically acceptable buffers, solvents, and surfactants.

In some cases, a genistein composition can include PVP (e.g., 5% PVP-K17) and polysorbate 80 (e.g., 0.2% polysorbate 80), as well as phosphate buffered saline (PBS, e.g., 50 nM PBS) and one or more chelating agents (e.g., ethylenediaminetetraacetic acid; EDTA). In some cases, for example, an oral formulation of a genistein composition can contain PVP (e.g., PVP-K25), polysorbate 80 (TWEEN® 80), and one or more preservatives (e.g., methyl paraben, propyl paraben, benzyl alcohol, or any combination thereof). In addition, a composition can include a diluent such as a sodium chloride solution.

When a composition contains nanoparticulate genistein, the particle size distribution of the genistein nanoparticles can be, for example, d(0.5)≤0.70 microns (e.g., d(0.5)≤0.60 microns, d(0.5)≤0.50 microns, d(0.5)≤0.40 microns, d(0.5)≤0.30 microns, or d(0.5)≤0.20 microns). See, e.g., U.S. Pat. No. 8,551,530. It is to be noted that genistein formulations characterized as suspensions can contain a measurable amount of genistein dissolved in the suspension medium, depending on the carrier(s), excipient(s), and diluent(s) included in the suspension medium.

Genistein exhibits low to virtually no solubility in several pharmaceutically acceptable solvents, but some formulations of genistein (e.g., nanoparticulate suspensions of genistein) can provide a high concentration of genistein. For example, a suspension of nanoparticulate genistein can incorporate genistein in amounts ranging from about 100 mg/mL to about 500 mg/mL (e.g., ranges from about 100 mg/mL to about 400 mg/mL, about 150 mg/mL to about 350 mg/mL, about 200 mg/mL to about 400 mg/mL, about 250 mg/mL to about 350 mg/mL, about 275 mg/mL to about 325 mg/mL, about 300 mg/mL to about 450 mg/mL, or about 350 mg/mL to about 500 mg/mL, or amounts of about 100 mg/mL, about 150 mg/mL, about 200 mg/mL, about 250 mg/mL, about 275 mg/mL, about 300 mg/mL, about 325 mg/mL, about 350 mg/mL, about 375 mg/mL, about 400 mg/mL, about 450 mg/mL, or about 500 mg/mL). The relative amount of genistein included in such a suspension can be varied to yield a formulation having a desired total content of genistein. For example, a suspension formulation as described herein can include up to about 50% (w/w) genistein [e.g., about 50% (w/w), about 45% (w/w), about 40% (w/w), about 35% (w/w), about 30% (w/w), about 25% (w/w), about 20% (w/w), about 15% (w/w), about 10% (w/w), about 40% to about 50% (w/w), about 35% to about 45%, about 30% to about 40% (w/w), about 25% to about 35% (w/w), about 20% to about 30% (w/w), about 20% to about 35% (w/w), about 15% to about 35%, about 10% to about 30%, or about 10% to about 25%]. In some embodiments, nanoparticle genistein suspensions can provide increased bioavailability of genistein as compared to the bioavailability of genistein provided by solution formulations (e.g., solutions containing a pharmaceutically acceptable PEG solvent or containing larger sized genistein material). As described in U.S. Pat. No. 8,551,530, for example, the combination of high genistein loading and significantly increased bioavailability can provide advantages, such as facilitating administration of therapeutically effective amounts of genistein using much lower amounts of formulated drug substance, for example.

Genistein compositions can be formulated for administration by any suitable method, depending upon whether local or systemic treatment is desired and upon the area to be treated. For example, a genistein composition can be formulated for oral administration, parenteral administration (e.g., by subcutaneous, intrathecal, intraventricular, intramuscular, or intraperitoneal injection, or by intravenous drip), pulmonary administration (e.g., by inhalation or insufflation of powders or aerosols or a nebulized mist), or by a combination of routes such as oral and parenteral administration. Administration can be rapid (e.g., by injection) or can occur over a period of time (e.g., by slow infusion or administration of slow release formulations, such as from subcutaneous drug depots, slow short term intravenous injections, or slow release oral formulations).

Compositions and formulations for parenteral administration include, for example, sterile solutions (e.g., sterile aqueous solutions or suspensions) that also can contain buffers, diluents, and/or other suitable additives (e.g., penetration enhancers, carrier compounds and other pharmaceutically acceptable carriers). Compositions formulated for parenteral delivery can be manufactured according to standard methods to provide sterile compositions deliverable via, for example, intravenous injection or infusion, intravascular injection, subcutaneous injection, or intramuscular injection. A genistein formulation (e.g., a suspension of nanoparticulate genistein) can be prepared to have a viscosity suitable for the desired route of parenteral administration, and can be manufactured and packaged in any manner suited to the desired application, including, without limitation, as a formulation deliverable via intravenous injection or infusion, intravascular injection, subcutaneous injection, or intramuscular injection. In some embodiments, a formulation as described herein can be contained in one or more pre-filled syringes or auto-injectors prepared for administration of a given dose or range of doses of genistein.

Genistein compositions also can be formulated for oral administration. Compositions and formulations for oral administration include, for example, powders, granules, suspensions or solutions in water or non-aqueous media (e.g., suspensions of genistein nanoparticles in edible oil), capsules, gel caps, sachets, and tablets. In some cases, a genistein composition can be prepared as a liquid suspension that can be metered to deliver a desired dose, or can be incorporated into capsules (e.g., gelatin or soft capsules) suitable for delivery of liquid formulations. Formulations for oral administration also can be loaded into prefilled sachets or premetered dosing cups. In some cases, such genistein formulations also can include one or more pharmaceutically acceptable sweetening agents, preservatives, dyestuffs, flavorings, or any combination thereof. Genistein-containing compositions can be produced for oral administration in any suitable packaging. In some cases, for example, powdered genistein can be combined with one or more excipients and packaged into individual or bulk containers that can be provided to a subject, who then can combine the genistein formulation with a beverage (e.g., water) for drinking.

In some cases, a solid formulation for oral administration can be prepared by spray drying an aqueous formulation of genistein to generate a powder containing crystals or nanocrystals. In some cases, a solid formulation for oral administration can be prepared by spray drying a solution containing genistein and one or more organic solvents to generate an amorphous solid dispersion. In some cases, a solid formulation for oral administration can be prepared by hot melt extrusion of a solution containing genistein and a polymer. See, U.S. Provisional Application No. 63/092,838 for a discussion of such solid formulations of genistein.

In some cases, a composition can be formulated for pulmonary administration in the form of a mist, such as via a nebulizer. A nebulizer is a device that typically uses oxygen, compressed air, or ultrasonic power to break up a solution or suspension into small aerosol droplets that can be directly inhaled from the mouthpiece of the device. A nebulizer can be powered mechanically (e.g., by a user's pumping action or actuation of a spring to increase and then quickly decrease the air pressure in a container holding the composition), in which cases a volatile liquid (e.g., alcohol) may be added to the composition to facilitate the increase in pressure. In some cases, a nebulizer can be powered electrically, using a vibrating mesh or a compressor, or an oscillator that generates a high frequency ultrasonic wave to cause mechanical vibration of a piezoelectric element.

Genistein compositions useful in the methods described herein can further include any pharmaceutically acceptable genistein salts, esters, or salts of such esters, or any other genistein compound which, upon administration to an animal such as a human, is capable of providing (directly or indirectly) biologically active genistein or an active metabolite or residue thereof. Accordingly, for example, provided herein are pharmaceutically acceptable salts of genistein, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of genistein (e.g., salts that retain the desired biological activity of genistein without imparting undesired toxicological effects). Examples of pharmaceutically acceptable salts may include, for example, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), and salts formed with organic acids (e.g., glucuronic acid, acetic acid, citric acid, oxalic acid, palmitic acid, or fumaric acid). Depending on the route of administration, for example, genistein may be sulfated or in glucuronic acid form.

Compositions also can include other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the genistein within the composition. The formulations can be sterilized if desired.

In some cases, this document provides methods for using genistein compositions as countermeasures against the pulmonary effects of coronavirus infection. Such countermeasures can be used to prevent, reduce, or mitigate effects such as pneumonitis, pulmonary fibrosis, damage to the pulmonary system, degenerative effects on lung tissue, and even death. It is noted that the methods described herein also can be used as countermeasures against other indications and sources of lung injury. Such indications can include any condition that causes ARDS. The mechanical cause of ARDS is fluid leaked from the smallest blood vessels in the lungs into the tiny air sacs where blood is oxygenated. Normally, a protective membrane keeps this fluid in the vessels, but severe illness or injury can cause damage to the membrane, leading to the fluid leakage of ARDS. Underlying causes of ARDS include, without limitation, sepsis, inhalation of harmful substances (e.g., high concentrations of smoke or chemical fumes), aspiration or near-drowning episodes, severe pneumonia, head or chest injury, accidents such as falls or car crashes, pancreatitis, massive blood transfusions, burns, drugs that cause pneumonitis (e.g., some antibiotics, chemotherapy drugs, medications that maintain regular heartbeat, and overdoses of aspirin), repeated exposure to molds and/or bacteria, exposure to feathers or bird excrement, radiation treatment of the chest (e.g., for breast or lung cancer) or the whole body (e.g., in preparation for a bone marrow transplant), granulocyte colony-stimulating factor (G-CSF)-related pulmonary toxicity (e.g., in patients treated with NEUPOGEN®), and pneumonia caused by pathogens such as pneumococci, influenza, viruses, malaria, and mycoplasmas.

In some cases, this document provides methods for treating a subject (e.g., a mammal, such as a human, a non-human primate, a mouse, a rat, a sheep, a pig, or a dog) to prevent or reduce one or more effects of an inflammatory disorder or an immunological disorder (e.g., infection by a coronavirus, such as SARS-CoV-2). For example, a method can include identifying a subject as having an inflammatory disorder or an immunological disorder (e.g., based on symptoms or molecular diagnostic test), or as being at risk for developing an inflammatory or immunological disorder, and administering to the subject an amount of a genistein-containing composition effective to reduce or prevent adverse effects of the disorder (e.g., fibrosis). In some cases, a method can include identifying a subject as having a coronavirus infection (e.g., based on symptoms or molecular diagnostic test), as having been exposed to coronavirus, or as being at risk for exposure to coronavirus, and administering to the subject an amount of a genistein-containing composition effective to reduce or prevent adverse effects of the coronavirus on the lung (e.g., pneumonitis or fibrosis). Subjects at risk for exposure to coronavirus include, for example, family members or other individuals (e.g., health care workers and first responders) in contact with a person who may have a coronavirus infection, or in contact with a person who has been exposed to someone else with a coronavirus infection. Thus, in some cases, individuals can be treated with a genistein-containing composition prophylactically, before exposure to coronavirus or another agent that may cause an inflammatory or immunological disorder, or after exposure but before the onset of symptoms indicative of infection. In some cases, for example, individuals can be treated with a genistein-containing composition after they present with symptoms that are consistent with a coronavirus infection (e.g., COVID-19), or after they are diagnosed as having a coronavirus infection (e.g., COVID-19). Common symptoms of COVID-19 include fever, cough, and/or shortness of breath (dyspnea), but other symptoms include, for example, fatigue, headache, chills, sore throat, lost sense of taste or smell, runny nose, body aches, and diarrhea.

In some cases, a subject can be an individual identified as having an inflammatory disorder or an immunological disorder (e.g., a coronavirus infection). Such subjects can be treated on an hourly, daily, or weekly basis after being identified as having a coronavirus infection. In some cases, a genistein composition can be administered within about four days or less (e.g., within about 96 hours, within about 72 to about 96 hours, about 48 to about 72 hours, about 24 to about 48 hours, about 20 to about 24 hours, about 18 to about 20 hours, about 16 to about 18 hours, about 12 to about 16 hours, about 8 to about 12 hours, about 6 to about 8 hours, about 4 to about 6 hours, about 2 to about 4 hours, about 1 to about 2 hours, or within about 60 minutes) after diagnosis or after onset of one or more symptoms. In some cases, a genistein composition can be administered starting four days or more after diagnosis or onset of symptoms. For example, a genistein composition can be administered beginning about four to seven days, about seven to about 14 days, about two to about four weeks, about four to about eight weeks, about eight to about 12 weeks, about 12 to about 14 weeks, about 14 to about 18 weeks, about 18 to about 22 weeks, about 22 to about 26 weeks, or more than 26 weeks after diagnosis or onset of symptoms. In some cases, a genistein composition can be administered to a patient who has been hospitalized and released, or to a mammal who tested positive for COVID-19 and has recovered (e.g., no longer has significant symptoms, no longer needs to quarantine, and/or no longer tests positive for the virus), in order to reduce or prevent long term effects (e.g., pulmonary fibrosis and/or reduced pulmonary function) as the patient recuperates. For example, a genistein composition can be administered beginning about one to seven days, about seven to about 14 days, about two to about four weeks, about four to about eight weeks, about eight to about 12 weeks, about 12 to about 14 weeks, about 14 to about 18 weeks, about 18 to about 22 weeks, about 22 to about 26 weeks, or more than 26 weeks after release from the hospital or recovery from COVID-19. Administration can continue on an hourly, daily, weekly, or monthly basis to mitigate the effects of the coronavirus. For example, a genistein-containing composition can be administered one or more times daily, every other day, biweekly, weekly, bimonthly, monthly, or less often, for any suitable length of time after infection by the virus has been identified (e.g., for about a week, about two weeks, about three weeks, about a month, about six weeks, about two months, about three months, about six months, about a year, or more than a year after diagnosis).

In some cases, a subject can be an individual exposed to a coronavirus (e.g., in a medical setting such as a clinic or a hospital, in another public setting, or in a non-public setting such as a household with a person having a coronavirus infection). Subjects exposed to coronavirus can be treated on an hourly, daily, or weekly basis at any time after exposure, in order to mitigate harmful effects of exposure to the virus. In some embodiments, a genistein composition can be administered within about four days or less (e.g., within about 96 hours, within about 72 to about 96 hours, about 48 to about 72 hours, about 24 to about 48 hours, about 20 to about 24 hours, about 18 to about 20 hours, about 16 to about 18 hours, about 12 to about 16 hours, about 8 to about 12 hours, about 6 to about 8 hours, about 4 to about 6 hours, or about 2 to about 4 hours), or within about 60 minutes or less (e.g., within about 45 to about 60 minutes, about 30 to about 45 minutes, about 15 to about 30 minutes, about 10 to about 15 minutes, or about 5 to about 10 minutes) after exposure. Administration can continue on an hourly, daily, weekly, or monthly basis to mitigate the effects of exposure to the coronavirus. For example, a genistein-containing composition can be administered one or more times daily, every other day, biweekly, weekly, bimonthly, monthly, or less often, for any suitable length of time after exposure to the virus has occurred (e.g., for about a week, about two weeks, about three weeks, about a month, about six weeks, about two months, about three months, about six months, about a year, or more than a year after exposure).

In some cases, an individual can be treated on a daily or weekly basis (e.g., every day, about six days per week, about five days per week, about four days per week, about three days per week, or about two days per week), before potential exposure to coronavirus. In some embodiments, an individual can be treated beginning within about 1 hour to about 14 days (e.g., with about 7 to about 14 days, about 6 to about 7 days, about 5 to about 6 days, about 4 to about 5 days, about 3 to about 4 days, about 60 to about 72 hours, about 48 to about 60 hours, about 36 to about 48 hours, about 24 to about 36 hours, about 18 to about 24 hours, about 12 to about 18 hours, about 10 to about 12 hours, about 8 to about 10 hours, about 6 to about 8 hours, about 4 to about 6 hours, about 2 to about 4 hours, or about 1 to about 2 hours) before potential exposure, such as before entering an area of increased risk for exposure to coronavirus (e.g., a clinical or hospital setting), in order to prevent or reduce potential harmful effects, should such exposure occur.

The methods provided herein include administering to a subject a composition that contains genistein in any formulation suitable to deliver an effective amount of the genistein to the subject, where the amount is effective to prevent or reduce one or more adverse events (e.g., tissue fibrosis) in the subject. As used herein, an amount that is “effective to reduce” one or more adverse effects of an inflammatory or immunological disorder in a subject is an amount that is sufficient to decrease one or more effects of disorder (e.g., fibrosis) as compared to the effect of the disorder observed before administration of genistein, or as compared to the level in a corresponding subject to which genistein was not administered. Fibrosis can be evaluated by, for example, computerized tomography (CT) scanning, spirometry, diffusing capacity for carbon monoxide (DLCO) or plethysmography. In some cases, an effective dose can prevent development of one or more adverse effects of an inflammatory or immunological disorder. Effective doses (e.g., therapeutically or prophylactically effective doses) can be effective to prevent, reduce, or mitigate effects of exposure to an agent (e.g., a virus or a bacteria) that can lead to an inflammatory or immunological response.

In some cases, the methods provided herein can include administering to a subject a composition that contains genistein in any formulation suitable to deliver an effective amount of the genistein to the subject, where the amount is effective to prevent or reduce one or more adverse pulmonary events (e.g., coronavirus-related pneumonitis and/or pulmonary fibrosis) in the subject. As used herein, an amount that is “effective to reduce” one or more adverse pulmonary effects of coronavirus in a subject is an amount that is sufficient to decrease one or more effects of infection (e.g., pneumonitis and/or pulmonary fibrosis) as compared to the effect of infection observed before administration of genistein, or as compared to the level in a corresponding subject to which genistein was not administered. Pneumonitis and pulmonary fibrosis can be evaluated by, for example, computerized tomography (CT) scanning, spirometry, DLCO, or plethysmography. In some cases, an effective amount of a genistein composition can be an amount that results in a reduced need for exogenous oxygen in the patient (e.g., an amount that leads to a reduction in the amount of exogenous oxygen required to maintain sufficient blood oxygenation in a patient). In some cases, an effective dose can prevent development of one or more adverse pulmonary effects of the coronavirus. Effective doses (e.g., therapeutically or prophylactically effective doses) can be effective to prevent, reduce, or mitigate effects of exposure to a coronavirus (e.g., SARS-CoV-2) that include, without limitation, pneumonitis, pulmonary fibrosis, pneumonia, dyspnea, pulmonary edema, and death.

In some embodiments, a therapeutic or prophylactic dose of a genistein-containing composition for administration to a human can contain about 0.25 g to about 5 g of genistein (e.g., about 0.25 g to about 0.3 g, about 0.3 g to about 0.4 g, about 0.4 g to about 0.5 g, about 0.5 g to about 0.75 g, about 0.75 g to about 1 g, about 1 g to about 1.25 g, about 1.25 g to about 1.5 g, about 1.5 g to about 1.75 g, about 1.75 g to about 2 g, about 2 g to about 2.25 g, about 2.25 g to about 2.5 g, about 2.5 g to about 3 g, about 3 g to about 4 g, or about 4 g to about 5 g). In some cases, an amount of a genistein-containing composition can be effective to achieve a blood concentration of aglycone (unconjugated, non-glucuronidated) genistein in a human of about 10 nM to about 10 μM (e.g., about 10 nM to about 25 nM, about 25 nM to about 50 nM, about 50 nM to about 75 nM, about 75 nM to about 100 nm, about 88 nM to about 880 nM, about 88 nM to about 150 nm, about 100 nM to about 200 nM, about 200 nM to about 500 nM, about 500 nM to about 800 nM, about 750 nM to about 1 μM, about 1 μM to about 2 μM, about 2 μM to about 5 μM, or about 5 μM to about 10 μM).

In some cases, a genistein composition can be administered to a subject (e.g., a human) at a dose of about 2.5 mg/kg to about 1 g/kg (e.g., about 2.5 to about 5 mg/kg, about 5 to about 10 mg/kg, about 10 to about 25 mg/kg, about 25 to about 50 mg/kg, about 50 mg/kg, about 75 to about 100 mg/kg, about 100 to about 200 mg/kg, about 200 to about 300 mg/kg, about 300 to about 400 mg/kg, about 400 to about 500 mg/kg, about 500 to about 750 mg/kg, or about 750 mg/kg to about 1 g/kg). The dose can be administered on a daily to weekly basis or longer (e.g., for about 1 day to about 2 days, such as from about 1 to about 3 days, about 3 to about 7 days, about 7 to about 10 days, about 10 to about 14 days, about 14 to about 21 days, about 21 to about 28 days, about 28 to about 35 days, about 30 to about 45 days, about 45 to about 60 days, about two to about three months, about three to about six months, about six to about nine months, or about nine months to about a year).

The administering step can be accomplished via any suitable route. In some embodiments, for example, a genistein composition containing a solution of genistein or a suspension of genistein nanoparticles can be administered orally or parenterally (e.g., by injection, such as subcutaneous, intravenous, or intramuscular injection).

In some embodiments, a therapeutic method can include administering a first dose of genistein for a first period of time after exposure (or potential exposure) to an agent that can cause an inflammatory or immunological response (e.g., a coronavirus) or after diagnosis with an inflammatory or immunological disorder (e.g., a coronavirus infection), and then administering a second dose of genistein for a second period of time. The first dose can be higher than the second dose. For example, a method can include administering a genistein-containing composition to a human at a dose of 0.25 g to about 5 g per day (e.g., about 0.25 g to about 0.3 g, about 0.3 g to about 0.4 g, about 0.4 g to about 0.5 g, about 0.5 g to about 0.75 g, about 0.75 g to about 1 g, about 1 g to about 1.25 g, about 1.25 g to about 1.5 g, about 1.5 g to about 1.75 g, about 1.75 g to about 2 g, about 2 g to about 2.25 g, about 2.25 g to about 2.5 g, about 2.5 g to about 3 g, about 3 g to about 4 g, or about 4 g to about 5 g per day) for about 1 day to about 2 weeks (e.g., about 1 day to about 3 days, about 3 days to about 7 days, about 7 days to about 10 days, or about 10 days to about 14 days), and then administering the composition at a dose of about 0.1 g to about 1 g per day (e.g., about 0.1 g to about 0.7 g, about 0.2 g to about 0.5 g, about 0.3 g to about 1 g, about 0.5 g to about 0.8 g, or about 0.5 g to about 1 g) for about 1 day to about 2 months (e.g., about 1 to about 3 days, about 3 to about 7 days, about 7 to about 10 days, about 10 to about 14 days, about 14 to about 21 days, about 21 to about 28 days, about 28 to about 35 days, about 30 to about 45 days, about 45 to about 60 days, or about two to about three months).

If a subject fails to respond to a particular amount of genistein, then the amount of the administered genistein composition can be increased by, for example, two fold. After receiving this higher amount, the subject can be monitored for both responsiveness to the treatment and toxicity symptoms, and further adjustments can be made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a genistein composition can be any frequency that reduces inflammatory injury (e.g., fibrosis or pneumonitis) without producing significant toxicity to the subject. For example, the frequency of administration can be from about once an hour to about once a week (e.g., from about four times daily to twice daily, about twice daily to once daily, about four times a week to twice a week, or about twice a week to once a week). The frequency of administration can remain constant or can be variable during the duration of treatment. A course of treatment with a composition containing genistein can include rest periods. In some cases, a composition containing genistein can be administered daily over a week-long period, followed by a rest period of one to seven days, and such a regimen can be repeated multiple times. As with the effective amount, various factors can influence the actual frequency of administration used for a particular application. In some cases, the effective amount, duration of treatment, use of multiple treatment agents, route of administration, and severity of the condition may require an increase or decrease in administration frequency.

An effective duration for administering a composition containing genistein can be any duration that alleviates or reduces inflammatory injury (e.g., fibrosis or pneumonitis) without producing significant toxicity to the subject, or any duration during which a subject may be at risk of exposure to an agent that may cause inflammation or an immune response (e.g., a virus or a bacteria). In some cases, the effective duration can vary from several days to several months (e.g., from about 4 to about 7 days, from about 7 to about 14 days, from about 1 to about 12 weeks, from about 2 to about 4 weeks, from about 4 to about 8 weeks, from about 8 to about 12 weeks, or more than 12 weeks). Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated can be monitored. Any appropriate method can be used to determine whether or not inflammation in a subject has been reduced. For example, a subject can be assessed by CT scan, spirometry, DLCO, or plethysmography after administration of genistein to determine if the treatment reduced the amount of tissue or organ injury (e.g., fibrosis), as compared to the level of injury observed in the subject prior to treatment. In some cases (e.g., with coronavirus infection), a subject can be assessed after administration of genistein to determine if the treatment reduced the amount of pneumonitis and/or pulmonary fibrosis in the subject, as compared to the level of pneumonitis and/or pulmonary fibrosis observed in the subject prior to treatment.

This document also provides for the use of genistein compositions as described herein for preventing, reducing, or mitigating one or more effects of an inflammatory or immunological disorder or exposure to an agent that may cause such a disorder (e.g., a virus or a bacteria) in a subject identified as having been infected by or exposed to the agent, or as being at risk of exposure to the agent. In addition, this document provides for the use of genistein in the manufacture of medicaments for preventing, reducing, or mitigating one or more effects of infection by or exposure to an agent capable of causing an inflammatory or immunological response in a subject identified as having an inflammatory or immunological disorder, as having been exposed to the agent, or as being at risk of exposure to the agent.

In some cases, this document also provides for the use of genistein compositions as described herein for preventing, reducing, or mitigating one or more effects of infection by or exposure to a coronavirus (e.g., SARS-CoV-2) in a subject identified as having been infected by or exposed to the coronavirus, or as being at risk of exposure to the coronavirus. In addition, this document provides for the use of genistein in the manufacture of medicaments for preventing, reducing, or mitigating one or more effects of infection by or exposure to coronavirus in a subject identified as having a coronavirus infection, as having been exposed to coronavirus, or as being at risk of exposure to coronavirus.

In addition, genistein formulations (e.g., genistein solutions or nanoparticle suspensions) can be combined with packaging material and sold as kits for preventing, reducing, or mitigating the effects of an inflammatory or immunological disorder (e.g., the effects of infection by or exposure to coronavirus). Thus, this document also provides articles of manufacture that can include one or more genistein-containing compositions. The articles of manufacture can further include, for example, buffers or other control reagents for reducing, preventing, or monitoring the effects of an inflammatory or immunological disorder (e.g., COVID-19) or exposure to an agent that can cause an inflammatory or immunological disorder (e.g., a bacteria or a virus, such as a coronavirus). Instructions describing how genistein formulations are effective for preventing, reducing, or mitigating damage from such exposure also can be included in such kits.

In some embodiments, an article of manufacture can include a genistein formulation (e.g., a suspension of nanoparticulate genistein) contained within a means for administration, such as an auto-injector or a nebulizer. For example, an auto-injector or nebulizer can contain a suspension of nanoparticulate genistein at a concentration between about 250 mg/mL and about 500 mg/mL, where the genistein nanoparticulate composition has a particle size distribution characterized by a d(0.5) of 0.5 μm or less (e.g., 0.4 μm or less, 0.3 μm or less, or 0.2 μm or less). The genistein composition also can include other components (e.g., one or more pharmaceutically acceptable excipients), as described herein.

Components and methods for producing articles of manufacture include those known in the art, for example. In addition, in some embodiments, pre-made auto-injectors and nebulizers can be obtained commercially, filled with a genistein nanoparticulate composition, and packaged as a kit for treating the effects of respiratory distress syndromes (e.g., pneumonitis, pulmonary fibrosis, pneumonia, dyspnea, and/or pulmonary edema).

The invention will be further described in the following examples, which do not limit the scope of the invention described in the claims.

### EXAMPLES

### Example 1—Efficacy of Genistein for Reducing Lung Fibrosis

Human lung tissue is known to undergo extensive degenerative remodeling following acute radiation exposure. This pathology, referred to as Delayed Effects of Acute Radiation Exposure in lung (DEARE-lung), results in shortness of breath, fatigue, and even death, all of which are caused by pneumonitis and pulmonary fibrosis. Analogous effects are observed in lung cancer patients receiving radiotherapy, because the radiation exposure can damage surrounding healthy tissue in addition to the tumor tissue. Mechanistically, the pathophysiology of DEARE-lung is similar to virally induced ARDS, in the sense that damage to the lung epithelium and endothelium results in an excessive immune response that further compounds tissue damage.

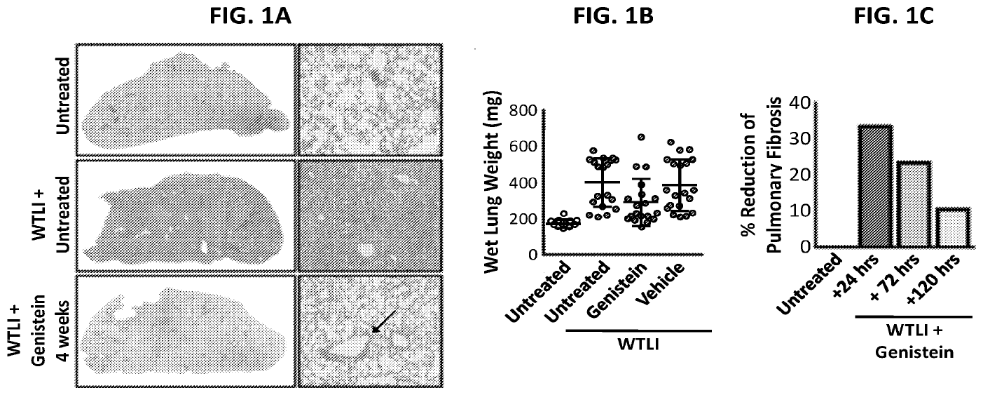

Studies using an oral suspension of nanoparticulate genistein demonstrated efficacy in a mouse model of DEARE-lung. In these studies, mice received a single dose of whole thoracic lung irradiation (WTLI), followed by daily treatment with the genistein composition beginning 24 hours after irradiation. Animals were treated for two, four, or six weeks. As compared to controls, the animals treated with genistein had significantly less immune infiltrate, edema, and fibrosis of the lungs as determined by histopathology and wet lung weight (FIGS. 1A and 1B). A reduction in pulmonary fibrosis was observed when genistein treatment started within five days of radiation, but the sooner treatment started, the better the results (FIG. 1C).

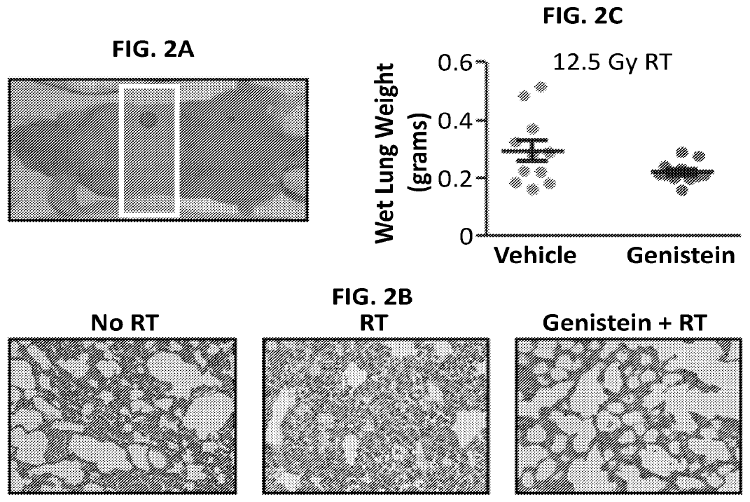

Other nonclinical studies focused on protection of the lungs in the context of cancer radiotherapy. A549 lung cancer cells were implanted subcutaneously in athymic CD1 nu/nu mice. The location of the implant was specific, so that a single dose of WTLI would hit the tumor and the normal lung tissue (FIG. 2A). Mice were then administered a nanoparticulate genistein suspension or vehicle control by daily oral gavage for seven days prior to WTLI and continuing until the end of the study. These studies showed that mice treated with genistein had a marked reduction in normal lung tissue remodeling (reduced thickening of the septal wall), as well as reduced congestion and minimal cellular infiltrate, as indicated by thin tissue sectioning and staining with hematoxylin and eosin (FIG. 2B). In addition, a reduction of pneumonitis was confirmed by reduced wet lung weight in genistein-treated animals, compared to animals treated with vehicle (FIG. 2C).

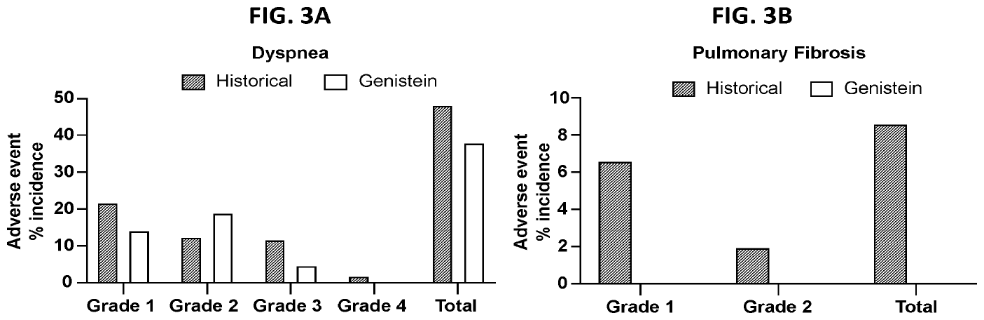

The use of genistein oral suspension also was studied in NSCLC patients. The drug was added to patients' chemoradiotherapy regimens to reduce the incidence of therapy-induced pneumonitis and pulmonary fibrosis. In particular, patients were given oral syringes preloaded with genistein oral suspension, so they could self-administer the drug at home. Patients ingested the suspension once daily for one week prior to chemoradiotherapy, and also while on concurrent chemoradiotherapy for a total of six to eight weeks. No dose-limiting toxicities were observed in any of the three dosing cohorts (500 mg/day, 1000 mg/day and 1500 mg/day). Pharmacokinetic evaluation demonstrated that all three doses levels produced serum levels of (biologically active) genistein aglycone that were sufficient to activate ERβ, although even at the highest dose (1500 mg/day), Cₘₐₓ levels were not adequate to reach the predicted EC₅₀ for estrogen receptor alpha (ERα). This was consistent with studies showing that genistein preferentially binds to ERβ over ERα (Landauer et al., J Rad Res 60(3):308-317, 2019). Interim data analysis was compared to a historical trial (Bradley et al., supra) that used the same chemoradiotherapy regimen but without genistein. The analysis showed that patients had a lower incidence of hematological, pulmonary, and gastrointestinal adverse events when treated with the nanoparticulate genistein oral suspension, and that such incidences were less severe in the genistein-treated patients. This included dyspnea, which had a lower incidence rate in genistein-treated patients (FIG. 3A), and pulmonary fibrosis, which 9% of patients in the historical study developed, but which none of the 21 patients treated with genistein have developed to date (FIG. 3B).

### Example 2—Effectiveness of Genistein in SARS-CoV Models

Several animal models have been used to study SARS-CoV infection; these include ferrets, nonhuman primates (commonly African green monkeys), and mice. SARS-CoV produces large viral titers in ferrets, but there is conflicting evidence that infection in ferrets demonstrates clinical symptoms of lung injury, so these models are thought to be best used for vaccine development (Venkataraman and Frieman, supra; and Gretebeck et al., Curr Opin Virol 13:123-129, 2015). Nonhuman primates produce similar clinical signs and viral titers as humans. In addition, a variety of mouse models exist for SARS-CoV. The most promising model that is applicable to SARS-CoV-2 research is the K18-hACE2 transgenic mouse (McCray et al., J Virol 81(2):813-821, 2007). This transgenic model uses the K18 promoter to drive expression of human ACE2, which is the host receptor through which SARS-CoV and SARS-CoV-2 gain entry into cells. Studies using this model for SARS research demonstrated that it produces a pathology similar to that observed in humans, and that it involves substantial pulmonary injury (Dediego et al., Virol 376(2):379-389, 2008; and McCray, supra). Another study using a transgenic mouse expressing human ACE2 showed infection-induced weight loss and also showed signs of pneumonia and pulmonary fibrosis, which were detected within 3-5 days post infection (Bao et al., supra). Thus, it is anticipated that the mouse model also produces a SARS-CoV-2 pathology similar to that observed in humans.

Given the above, the mouse model is used to test the efficacy of genistein for preventing lung injury due to SARS-CoV-2 infection. Without being bound by a particular mechanism, it is believed that genistein treatment can prevent infection-induced pneumonia and pulmonary fibrosis by inhibiting the virus-induced inflammatory response, leading to increased survival and decreased weight loss in the animals.

Mice are treated according to the schedule in TABLE 1A or TABLE 1B:

TABLE 1A
K18-hACE2 Mouse Study 1

| | Test Article | Dose Level | | | No. of |
|---|---|---|---|---|---|
| Group | Treatment | (mg/kg/dose) | Route | Schedule | Animals |
| 1 | Vehicle | — | Oral | QD, 2 Wk | 15-20 |
| 2 | Genistein | 200 | Oral | QD, 2 Wk | 15-20 |
| 3 | Genistein | 400 | Oral | QD, 2 Wk | 15-20 |
| 4 | Genistein | 800 | Oral | QD, 2 Wk | 15-20 |

TABLE 1B
K18-hACE2 Mouse Study 1

| | Test Article | Dose Level | | | No. of |
|---|---|---|---|---|---|
| Group | Treatment | (mg/kg/dose) | Route | Schedule | Animals |
| 1 | Vehicle | — | Oral | BID, 1 Wk | 15-20 |
| 2 | Genistein | 100 | Oral | BID, 1 Wk | 15-20 |
| 3 | Genistein | 200 | Oral | BID, 1 Wk | 15-20 |
| 4 | Genistein | 400 | Oral | BID, 1 Wk | 15-20 |

Two mouse studies are used to evaluate genistein's efficacy against COVID-19. Study 1 is conducted to quickly identify signs of efficacy and determine the optimal dose level. Mice are challenged with a molecular clone of SARS-CoV-2 through intranasal inoculation and then randomized into one of four treatment groups (TABLE 1A or TABLE 1B). Based on previous SARS studies with this model, lung injury is likely to occur in vehicle-treated mice within three to five days post infection (dpi), and mice are likely to succumb to disease in seven to ten days dpi. Mice are treated with genistein or vehicle starting two hours after infection. In the clinic, patients would presumably be treated at the onset of symptoms, but since disease progresses significantly faster in mice than in humans, treatment in mice is initiated shortly after infection. Genistein or vehicle is administered by oral gavage daily (QD) or twice daily (BID) within about two hours after infection, and administration is continued for 7 to 14 days. Animals that survive 14 days are euthanized for histopathological analysis. The genistein dose levels in Groups 2 and 3 are based on levels that showed efficacy in previous studies (FIGS. 1A-1C and 2A-2C). Additionally, dose levels 200 mg/kg and 400 mg/kg are roughly equivalent to the human doses 1000 mg and 1500 mg according to the FDA Center for Drug Evaluation and Research guidance for conversion of animal doses to human equivalent doses based on body surface area. In addition, the Group 3 dose level is included because the radiation-induced fibrosis efficacy studies involved at least four weeks of dosing, and since disease progresses so quickly in this model, a two-fold higher dose also was included. Endpoints for this experiment are survival and body weight, which are monitored daily and wet lung weight and lung histopathology at the time of death or euthanasia. This allows for expeditious completion and analysis of the study results so that the second mouse study design can be finalized and initiated. It is anticipated that treatment with genistein prevents pulmonary injury in this model, leading to an increase in survival.

For a second mouse study, animals are treated according to TABLE 2A or 2B:

TABLE 2A
K18-hACE2 Mouse Study 2

| | Test Article | Dose Level | | | No. of |
|---|---|---|---|---|---|
| Group | Treatment | (mg/kg/dose) | Route | Schedule | Animals |
| 1 | Vehicle | — | Oral | QD, 2 Wk | 15-20 |
| 2 | Genistein | ≥800 | Oral | QD, 2 Wk | 15-20 |
| 3 | Genistein | 200-400 | Oral | BID, 2 Wk | 15-20 |
| 4 | Vehicle | — | IM | Single Dose | 15-20 |
| 5 | Genistein | 200 | IM | Single Dose | 15-20 |

TABLE 2B
K18-hACE2 Mouse Study 2

| | Test Article | Dose Level | | | No. of |
|---|---|---|---|---|---|
| Group | Treatment | (mg/kg/dose) | Route | Schedule | Animals |
| 1 | Vehicle | — | Oral | BID, 1 Wk | 15-20 |
| 2 | Genistein | 100-400 | Oral | BID, 1 Wk | 15-20 |

In the second study, the same mouse model is utilized for a natural history study that is performed to gain additional confidence in positive results from the first study, or to optimize the dose level/schedule of genistein to improve efficacy (TABLE 2A or TABLE 2B). The dose level that produced positive results in Study 1 is repeated. In some cases, in the event that insufficient efficacy was observed in Study 1, several steps are taken in Study 2 to optimize drug dosing. First, a higher dose level is administered to test whether increased drug exposure promotes better efficacy (TABLE 2A, Group 2). Second, since several studies of genistein as a prophylactic treatment for radiation injury indicated that twice daily dosing (BID) may be superior to QD dosing, a dose of 200 to 400 mg/kg given BID is tested (TABLE 2A, Group 3). Third, a parenteral formulation of genistein (an injectable suspension) is tested (TABLE 2A, Group 5); this formulation showed efficacy in preventing radiation injury after just a single intramuscular (IM) dose. The IM route of administration had superior pharmacokinetics compared to the oral suspension, and therefore may provide improved efficacy over oral administration. If necessary, it is tested by administering a single IM dose two hours after infection. In addition, if sufficient efficacy is observed in the first study, the corresponding dose and regimen may be used in study 2 (TABLE 2B).

Survival and body weight are monitored to recapitulate previous findings. Furthermore, the study is powered appropriately so that mice can be sacrificed at appropriate time points (e.g., every three days, or on days 2, 4, 6, and 14) to monitor lung injury over time. This can be done by first collecting bronchoalveolar lavage specimens to analyze immune cell infiltrate in infected lungs using Diff-Quik stain as described elsewhere (McCray et al., supra). Next, signs of lung injury are examined following established protocols (Jackson et al., Int J Rad Oncol Blot Phys 105(2):400-409, 2019; and Jackson et al., Brit J Pharmacol 174(24):4738-4750, 2017) by removing lungs from euthanized animals and measuring wet lung weight as a sign of inflammation and fibrosis. Lungs then are fixed and sectioned for histopathological analysis by H&E and Masson Trichrome stain to look for histopathological signs of injury and collagen buildup, respectively.

### Example 3—Effectiveness of Genistein on Irradiated Heart Cells

Further studies using genistein were conducted in microHeart cells (StemoniX, Maple Grove, MN) exposed to different types of radiation. In a first set of studies, microHeart cells were treated with genistein (0.5 μM or 5 μM, with a vehicle control) 24 hours prior to exposure to γ radiation. Cell survival was evaluated 24 hours after radiation exposure. While 1 Gy γ-radiation exerted a killing effect on vehicle-treated cells, the killing effect was mitigated by genistein (FIG. 4A). In a second set of studies, microHeart cells were treated with 0.5 μM genistein or vehicle control 24 hours prior to exposure to mixed field radiation (˜32% neutron, −68% gamma). Cell survival was evaluated 72 hours after radiation exposure. While 1 Gy mixed field radiation exerted a killing effect on vehicle-treated cells, the killing effect was again mitigated by genistein (FIG. 4B). Thus, genistein was radioprotective of cardiac cells. Moreover, since cell death causes the release of pro-inflammatory signals, protecting cells from death may reduce inflammation.

### Example 4—Effectiveness of Genistein on Lung and Heart Tissue of Irradiated Mice

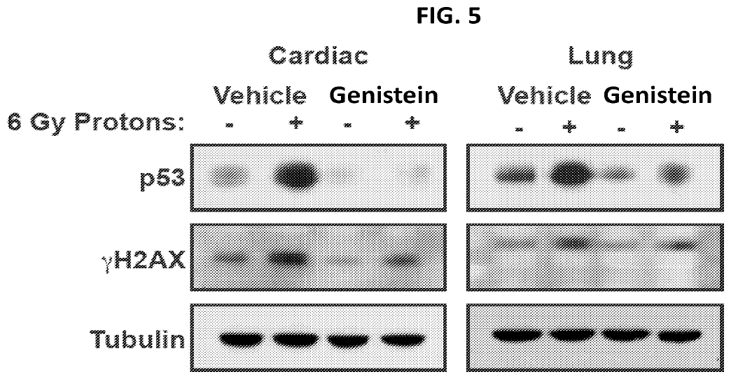

Mice were treated with a single subcutaneous injection of vehicle or nanoparticulate genistein (200 mg/kg) 24 hours prior to irradiation with 6 Gy high LET protons. Animals were sacrificed three days after irradiation, and total protein lysates were obtained from heart and lung tissue. Proteins in the lysates were quantitated using amido black staining, and standard western blotting was performed with antibodies against p53, γH2AX, and tubulin (FIG. 5). These results suggested protection of normal tissues at day three post-irradiation. Notably, the results were consistent between the two DNA damage markers, as elevated levels of γH2AX should be associated with increased levels of p53. Protection of normal tissue mediated through the DNA damage response can result in reduced cell death, and thus may also result in a reduction of the inflammatory response (via mitigation of cell death-dependent release of pro-inflammatory cytokines).

### Example 5—Effectiveness of Genistein on Pancreatitis

Acute pancreatitis is induced in Swiss albino mice by six hourly intraperitoneal injections of cerulein (50 μg/kg/hr). This model presents with pancreatic inflammation and cellular apoptosis, making it ideal for evaluating potential therapeutic interventions. Vehicle or nanoparticulate genistein (200 mg/kg) is administered prior to cerulein exposure to determine the extent of genistein's ability to prevent pancreatitis. Vehicle or nanoparticulate genistein (200 mg/kg) also is administered post cerulein exposure to determine the extent of genistein's ability to mitigate agent or a therapeutic. The degree of pancreatitis is measured by plasma levels of amylase and lipase, pancreatic edema as measured by the ratio of pancreas weight to body weight, histological analysis (necrosis, extent of edema, infiltration of inflammatory cells) of tissue sections following hematoxylin-eosin staining. Proinflammatory cytokine levels and other markers of pancreatic injury (NF-κB p65, SIRT1, TNF-α, iNOS, IL-1(3, IL-6, nitrotyrosine, Bax, Bcl2, caspase 3) can be measured using pancreatic tissue lysates. Nanoparticulate genistein-mediated reduction in one or more proinflammatory cytokines, markers of pancreatic injury, plasma amylase and/or lipase, or improvement detected with histological analyses supports its therapeutic potential.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. Use of a composition comprising genistein to reduce inflammation and/or an immune response in a mammal identified as having an inflammatory or immunological disorder.

2. The use of claim 1, wherein the genistein is nanoparticulate genistein.

3. The use of claim 2, wherein the composition has a nanoparticulate genistein concentration between about 250 mg/mL and about 500 mg/mL.

4. The use of any one of claims 1 to 3, wherein the mammal is a human.

5. The use of any one of claims 1 to 4, wherein the mammal was identified as having an inflammatory disorder selected from the group consisting of pancreatitis, myocarditis, and acute respiratory distress syndrome.

6. The use of any one of claims 1 to 4, wherein the inflammatory or immunological disorder is not COVID-19.

7. The use of any one of claims 1 to 6, wherein the composition comprises nanoparticulate genistein with a particle size distribution characterized by a d(0.5) less than or equal to 0.5 μm.

8. The use of any one of claims 1 to 7, wherein the composition further comprises one or more pharmaceutically acceptable excipients forming a suspension medium, wherein the one or more pharmaceutically acceptable excipients include a water soluble polymer comprising a polyvinylpyrrolidone.

9. The use of claim 8, wherein the one or more pharmaceutically acceptable excipients include a nonionic surfactant, a diluent, or a buffer.

10. The use of claim 9, wherein the composition comprises a nonionic surfactant, and wherein the nonionic surfactant is present in an amount ranging from about 0.01% to about 10% by weight (w/w).

11. The use of claim 8, wherein the amount of water soluble polymer is about 0.5% to about 15% (w/w).

12. The use of any one of claims 1 to 11, wherein the composition comprises a diluent and a preservative.

13. The use of claim 12, wherein the composition further comprises a non-ionic surfactant.

14. The use of any one of claims 1 to 13, wherein the composition comprises nanoparticulate genistein in an amount ranging up to about 50% (w/w).

15. The use of any one of claims 1 to 13, wherein the composition comprises nanoparticulate genistein in an amount of about 20% to about 35% (w/w).

16. The use of any one of claims 1 to 15, wherein the composition comprises nanoparticulate genistein at a concentration of about 325 mg/mL.

17. The use of any one of claims 1 to 16, wherein the composition has a pH of about 2 to about 12.

18. The use of any one of claims 1 to 17, wherein the composition is formulated as a tablet, a capsule, a gel cap, a powder, or a liquid suspension.

19. The use of any one of claims 1 to 18, wherein the composition is formulated for oral, intramuscular, subcutaneous, or intravenous administration.

20. The use of any one of claims 1 to 19, wherein the composition is for use within about 24 hours to about 7 days of diagnosis of the mammal as having the inflammatory or immunological disorder, or within about 24 hours to about 7 days of onset of one or more symptoms of the inflammatory or immunological disorder.

21. The use of any one of claims 1 to 20, wherein the composition is for use at least once daily.

22. The use of any one of claims 1 to 21, wherein the composition is for use for about 1 week to about 12 weeks.

23. The use of any one of claims 1 to 22, wherein the composition is formulated for administration in an amount of about 0.5 g to about 2.5 g per dose.

24. The use of any one of claims 1 to 22, wherein the composition is formulated for administration in an amount of about 1 g to about 1.5 g per dose.

25. A method for reducing inflammation and/or an immune response in a mammal identified as having an inflammatory or immunological disorder, the method comprising administering to the mammal a composition comprising genistein in an amount effective to reduce inflammation and/or an immune response in the mammal.

26. The method of claim 25, wherein the genistein is nanoparticulate genistein.

27. The method of claim 26, wherein the composition has a nanoparticulate genistein concentration between about 250 mg/mL and about 500 mg/mL.

28. The method of any one of claims 25 to 27 wherein the mammal is a human.

29. The method of any one of claims 25 to 28, wherein the mammal was identified as having an inflammatory disorder selected from the group consisting of pancreatitis, myocarditis, and acute respiratory distress syndrome.

30. The method of any one of claims 25 to 28 wherein the inflammatory or immunological disorder is not COVID-19.

31. The method of any one of claims 25 to 30 wherein the composition comprises nanoparticulate genistein with a particle size distribution characterized by a d(0.5) less than or equal to 0.5 μm.

32. The method of any one of claims 25 to 31, wherein the composition further comprises one or more pharmaceutically acceptable excipients forming a suspension medium, wherein the one or more pharmaceutically acceptable excipients include a water soluble polymer comprising a polyvinylpyrrolidone.

33. The method of claim 32, wherein the one or more pharmaceutically acceptable excipients include a nonionic surfactant, a diluent, or a buffer.

34. The method of claim 33, wherein the composition comprises a nonionic surfactant, and wherein the nonionic surfactant is present in an amount ranging from about 0.01% to about 10% by weight (w/w).

35. The method of claim 32, wherein the amount of water soluble polymer is about 0.5% to about 15% (w/w).

36. The method of any one of claims 25 to 35, wherein the composition comprises a diluent and a preservative.

37. The method of claim 36, wherein the composition further comprises a non-ionic surfactant.

38. The method of any one of claims 25 to 37, wherein the composition comprise nanoparticulate genistein in an amount ranging up to about 50% (w/w).

39. The method of any one of claims 25 to 37, wherein the composition comprises nanoparticulate genistein in an amount of about 20% to about 35% (w/w).

40. The method of any one of claims 25 to 39, wherein the composition comprises nanoparticulate genistein at a concentration of about 325 mg/mL.

41. The method of any one of claims 25 to 39, wherein the composition has a pH of about 2 to about 12.

42. The method of any one of claims 25 to 41, wherein the composition is formulated as a tablet, a capsule, a gel cap, a powder, or a liquid suspension.

43. The method of any one of claims 25 to 41, comprising administering the composition orally, intramuscularly, subcutaneously, or intravenously.

44. The method of any one of claims 25 to 43, comprising administering the composition within about 24 hours to about 7 days of diagnosis of the mammal as having the inflammatory or immunological disorder, or within about 24 hours to about 7 days of onset of one or more symptoms of the inflammatory or immunological disorder.

45. The method of any one of claims 25 to 44, comprising administering the composition at least once daily.

46. The method of any one of claims 25 to 45, comprising administering the composition for about 1 week to about 12 weeks.

47. The method of any one of claims 25 to 46, comprising administering the composition in an amount of about 0.5 g to about 2.5 g per dose.

48. The method of any one of claims 25 to 46, comprising administering the composition in an amount of about 1 g to about 1.5 g per dose.
